(19) United States
(12) Patent Application Publication
(10) Pub. No.: US 2010/0292295 A1
(43) Pub. Date: Nov. 18, 2010

(54) 5-OXO-3-PYRROLIDINECARBOXAMIDE DERIVATIVES AS P2X7 MODULATORS

(75) Inventors: Jon Graham Anthony Steadman, Harlow (GB); Daryl Simon Walter, Essex (GB)

Correspondence Address: GlaxoSmithKline;GLOBAL PATENTS -US, UW2220, P. O. BOX 1539, KING OF PRUSSIA, PA, 19406-0939, US

(73) Assignee: GLAXO GROUP LIMITED, Greenford Middlesex (GB)

(21) Appl. No.: 12/808,017

(22) PCT Filed: Dec. 17, 2008
(86) PCT No.: PCT/EP2008/067733

§ 371 Date: Jun. 14, 2010

(30) Foreign Application Priority Data
Dec. 18, 2007 (GB) 0724623.4
Jan. 17, 2008 (GB) 0800839.3

(51) Int. Cl. A61K 31/4015; C07D 207/277; A61P 19/02; A61P 25/28; A61P 25/16; A61P 29/00

(52) U.S. Cl. 514/423; 548/537

(57) ABSTRACT

The present invention provides a compound of Formula (IA) or a pharmaceutically acceptable salt thereof:
wherein R¹ represents C₁₋₄ alkyl, C₂₋₆ alkenyl, C₂₋₆ alkynyl, C₃₋₆ cycloalkyl, C₃₋₆ cycloalkylmethyl-, phenyl-X— or heteroaryl, any of which may be optionally substituted; X represents —(CR¹²R¹³)ₙ—; n represents 0 to 2; and R⁷, R⁸, R⁹, R¹⁰ and R¹¹ independently represent H, halogen, or cyano; or optionally substituted C₁₋₆ alkyl, C₂₋₆ alkenyl, C₂₋₆ alkynyl or C₃₋₆ cycloalkyl; such that at least two of R⁷, R⁸, R⁹, R¹⁰ and R¹¹ represent a group other than H and at least one of R⁷ and R¹¹ represents a group other than H; and wherein the compound is other than N-[(2,4-dichlorophenyl)methyl]5-oxo-1-(phenylmethyl)-3-pyrrolidinecarboxamide. The compounds and salts are thought to modulate P2X7 receptor function and be capable of antagonizing the effects of ATP at the P2X7 receptor.

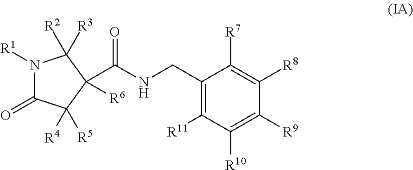

The present invention relates to 5-oxo-3-pyrrolidinecarboxamide derivatives which modulate P2X7 receptor function and are capable of antagonizing the effects of ATP at the P2X7 receptor (“P2X7 receptor antagonists”); to processes for their preparation; to pharmaceutical compositions containing them; and to the use of such compounds in therapy.

The P2X7 receptor is a ligand-gated ion-channel which is expressed in cells of the hematopoietic lineage, e.g. macrophages, microglia, mast cells, and lymphocytes (T and B) (see, for example, Collo, et al. Neuropharmacology, Vol. 36, pp 1277-1283 (1997)), and is activated by extracellular nucleotides, particularly adenosine triphosphate (ATP). Activation of P2X7 receptors has been implicated in giant cell formation, degranulation, cytolytic cell death, CD62L shedding, regulation of cell proliferation, and release of proinflammatory cytokines such as interleukin 1 beta (IL-1β) (e.g. Ferrari, et al., J. Immunol., Vol. 176, pp 3877-3883 (2006)) and tumour necrosis factor alpha (TNFα) (e.g. Hide, et al. Journal of Neurochemistry, Vol. 75, pp 965-972 (2000)). P2X7 receptors are also located on antigen presenting cells, keratinocytes, parotid cells, hepatocytes, erythrocytes, erythroleukaemic cells, monocytes, fibroblasts, bone marrow cells, neurones, and renal mesangial cells. Furthermore, the P2X7 receptor is expressed by presynaptic terminals in the central and peripheral nervous systems and has been shown to mediate glutamate release in glial cells (Anderson, C. et al. Drug. Dev. Res., Vol. 50, page 92 (2000)).

The localisation of the P2X7 receptor to key cells of the immune system, coupled with its ability to release important inflammatory mediators from these cells suggests a potential role of P2X7 receptor antagonists in the treatment of a wide range of diseases including pain and neurodegenerative disorders. Recent preclinical in vivo studies have directly implicated the P2X7 receptor in both inflammatory and neuropathic pain (Dell'Antonio et al., Neurosci. Lett., Vol. 327, pp 87-90 (2002), Chessell, I P., et al., Pain, Vol. 114, pp 386-396 (2005), Honore et al., J. Pharmacol. Exp. Ther., Vol. 319, p 1376-1385 (2006)) while there is in vitro evidence that P2X7 receptors mediate microglial cell induced death of cortical neurons (Skaper, S. D., et al., Glia, Vol. 54, p234-242 (2006)). In addition, up-regulation of the P2X7 receptor has been observed around β-amyloid plaques in a transgenic mouse model of Alzheimer's disease (Parvathenani, L. et al. J. Biol. Chem., Vol. 278(15), pp 13309-13317 (2003)).

The present invention provides compounds which modulate P2X7 receptor function and are capable of antagonizing the effects of ATP at the P2X7 receptor (P2X7 receptor antagonists).

In a first aspect of the invention, there is provided a pharmaceutical composition comprising a compound of formula (I) or a pharmaceutically acceptable salt thereof:

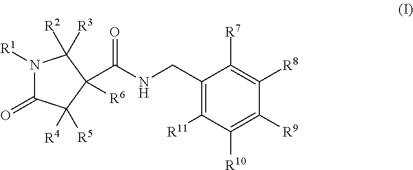

wherein:
R¹ represents C₁₋₄ alkyl, C₂₋₆ alkenyl, C₂₋₆ alkynyl, C₃₋₆ cycloalkyl, C₃₋₆ cycloalkylmethyl-, phenyl-X— or heteroaryl, any of which may be optionally substituted with C₁₋₆ alkyl, CF₃, —O—C₁₋₆ alkyl, CN or 1, 2 or 3 halogen (e.g. fluorine) atoms;
X represents —(CR¹²R¹³)ₙ—;
R¹² and R¹³ represent hydrogen or C₁₋₆ alkyl;
n represents an integer selected from 0 to 2;
R², R³, R⁴, R⁵ and R⁶ independently represent hydrogen, fluorine or methyl; and
R⁷, R⁸, R⁹, R¹⁰ and R¹¹ independently represent hydrogen, halogen (e.g. fluorine or chlorine), cyano, C₁₋₆ alkyl (e.g. methyl), C₂₋₆ alkenyl, C₂₋₆ alkynyl, C₃₋₆ cycloalkyl, wherein any of said C₁₋₆ alkyl, C₂₋₆ alkenyl, C₂₋₆ alkynyl or C₃₋₆ cycloalkyl groups may be optionally substituted with 1, 2 or 3 halogen (e.g. fluorine) atoms,
such that at least two of R⁷, R⁸, R⁹, R¹⁰ and R¹¹ represent a group other than hydrogen and at least one of R⁷ and R¹¹ represents a group other than hydrogen.

In a second aspect of the invention, there is provided a compound of formula (IA) or a pharmaceutically acceptable salt thereof:

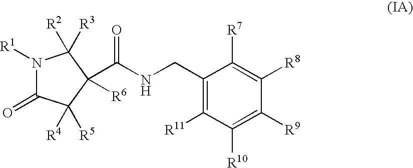

wherein:
R¹ represents C₁₋₄ alkyl, C₂₋₆ alkenyl, C₂₋₆ alkynyl, C₃₋₆ cycloalkyl, C₃₋₆ cycloalkylmethyl-, phenyl-X— or heteroaryl, any of which may be optionally substituted with C₁₋₆ alkyl, CF₃, —O—C₁₋₆ alkyl, CN or 1, 2 or 3 halogen (e.g. fluorine) atoms;
X represents —(CR¹²R¹³)ₙ—;
R¹² and R¹³ represent hydrogen or C₁₋₆ alkyl;
n represents an integer selected from 0 to 2;
R², R³, R⁴, R⁵ and R⁶ independently represent hydrogen, fluorine or methyl; and
R⁷, R⁸, R⁹, R¹⁰ and R¹¹ independently represent hydrogen, halogen (e.g. fluorine or chlorine), cyano, C₁₋₆ alkyl (e.g. methyl), C₂₋₆ alkenyl, C₂₋₆ alkynyl, C₃₋₆ cycloalkyl, wherein any of said C₁₋₆ alkyl, C₂₋₆ alkenyl, C₂₋₆ alkynyl or C₃₋₆ cycloalkyl groups may be optionally substituted with 1, 2 or 3 halogen (e.g. fluorine) atoms,
such that at least two of R⁷, R⁸, R⁹, R¹⁰ and R¹¹ represent a group other than hydrogen and at least one of R⁷ and R¹¹ represents a group other than hydrogen, with the proviso that the compound of formula (IA) is other than: N-[(2,4-dichlorophenyl)methyl]-5-oxo-1-(phenylmethyl)-3-pyrrolidinecarboxamide.

As used herein, the term “alkyl” (when used as a group or as part of a group) refers to a straight or branched hydrocarbon chain containing the specified number of carbon atoms. For example, C₁₋₆ alkyl means a straight or branched hydrocarbon chain containing at least 1 and at most 6 carbon atoms. Examples of alkyl include, but are not limited to: methyl (Me), ethyl (Et), n-propyl, i-propyl (isopropyl), n-butyl, i-butyl (isobutyl), sec-butyl, t-butyl, n-hexyl and i-hexyl.

As used herein, the term “alkenyl” refers to a straight or branched hydrocarbon chain containing the specified number of carbon atoms wherein at least once carbon-carbon bond is a double bond. Examples of alkenyl include, but are not limited to ethenyl, propenyl, n-butenyl, i-butenyl, n-pentenyl and i-pentenyl.

As used herein, the term “alkynyl” refers to a straight or branched hydrocarbon chain containing the specified number of carbon atoms wherein at least once carbon-carbon bond is a triple bond. Examples of alkynyl include, but are not limited to ethynyl, propynyl, butynyl, i-pentynyl, n-pentynyl, i-hexynyl and n-hexynyl.

The term ‘cycloalkyl’ unless otherwise stated means a closed 3 to 8 membered non-aromatic ring, for example cyclopropyl, cyclobutyl, cyclopentyl, cyclohexyl, cycloheptyl or cyclooctyl; in particular cyclopropyl, cyclobutyl, cyclopentyl or cyclohexyl.

The term ‘halogen’ is used herein to mean, unless otherwise stated, a group being fluorine, chlorine, bromine or iodine.

The term ‘heteroaryl’ as used herein refers to a 5-6 membered monocyclic aromatic or a fused 8-10 membered bicyclic aromatic ring containing 1 to 4 heteroatoms selected from oxygen, nitrogen and sulphur. Examples of such monocyclic aromatic rings include thienyl, furyl, furazanyl, pyrrolyl, triazolyl, tetrazolyl, imidazolyl, oxazolyl, thiazolyl, oxadiazolyl, isothiazolyl, isoxazolyl, thiadiazolyl, pyranyl, pyrazolyl, pyrimidyl, pyridazinyl, pyrazinyl, pyridyl, triazinyl, tetrazinyl and the like. Examples of such fused aromatic rings include quinolinyl, isoquinolinyl, quinazolinyl, quinoxalinyl, pteridinyl, cinnolinyl, phthalazinyl, naphthyridinyl, indolyl, isoindolyl, azaindolyl, indolizinyl, indazolyl, purinyl, pyrrolopyridinyl, furopyridinyl, benzofuranyl, isobenzofuranyl, benzothienyl, benzoimidazolyl, benzoxazolyl, benzoisoxazolyl, benzothiazolyl, benzoisothiazolyl, benzoxadiazolyl, benzothiadiazolyl and the like.

It is to be understood that the present invention covers and discloses all possible combinations of particular, preferred, suitable, or other embodiments of groups or features (e.g. of R¹, R², R³, R⁴, R⁵, R⁶, R⁷, R⁸, R⁹, R¹⁰, R¹¹, R¹², R¹³, X, and/or n), e.g. covers and discloses all possible combinations of embodiments of different groups or features, which embodiments are described herein.

In certain particular embodiments, R¹ represents C₁₋₄ alkyl (e.g. methyl, ethyl or isobutyl).

In certain particular embodiments, R¹ represents C₃₋₆ cycloalkyl (e.g. cyclopropyl). In a further particular embodiment, R¹ represents C₃₋₅ cycloalkyl (e.g. cyclopropyl).

In certain embodiments, R¹ represents phenyl-X— (e.g. phenyl-CH(Me)- or phenyl-CH₂—).

In certain embodiments X represents —CH(Me)- or —CH₂—.

In certain preferred embodiments, R¹ represents C₁₋₄ alkyl (e.g. methyl, ethyl or isobutyl) or C₃₋₅ cycloalkyl (e.g. cyclopropyl). For example, R¹ can represent methyl, ethyl, isobutyl or cyclopropyl. Preferably, R¹ represents methyl, ethyl or cyclopropyl.

In certain particular embodiments, R², R³, R⁴, R⁵ and R⁶ each represent hydrogen.

In certain particular embodiments, R⁷ represents hydrogen, halogen (e.g. fluorine or chlorine) or C₁₋₆ alkyl (e.g. methyl) optionally substituted with 1, 2 or 3 halogen (e.g. fluorine) atoms (e.g. —CF₃). Therefore, in one particular embodiment, R⁷ represents hydrogen, halogen (e.g. fluorine or chlorine), methyl or —CF₃. In a further particular embodiment, R⁷ represents halogen (e.g. fluorine or chlorine). Preferably, R⁷ represents chlorine.

In certain particular embodiments, R⁸ represents hydrogen or C₁₋₆ alkyl (e.g. methyl) optionally substituted with 1, 2 or 3 halogen (e.g. fluorine) atoms (e.g. —CF₃). In one particular embodiment, R⁸ represents hydrogen, methyl or —CF₃; more particularly hydrogen or —CF₃.

In certain particular embodiments, R⁹ represents hydrogen or halogen (e.g. fluorine or chlorine).

In certain particular embodiments, R¹⁰ represents hydrogen, halogen (e.g. fluorine or chlorine) or C₁₋₆ alkyl (e.g. methyl) optionally substituted with 1, 2 or 3 halogen (e.g. fluorine) atoms (e.g. —CF₃). Therefore, in one particular embodiment, R¹⁰ represents hydrogen, halogen (e.g. fluorine or chlorine), methyl or —CF₃; more particularly hydrogen, fluorine, chlorine or —CF₃. In a further particular embodiment, R¹⁰ represents hydrogen.

In certain particular embodiments, R¹¹ represents hydrogen, halogen (e.g. fluorine or chlorine) or C₁₋₆ alkyl (e.g. methyl) optionally substituted with 1, 2 or 3 halogen (e.g. fluorine) atoms (e.g. —CF₃). Therefore, in one particular embodiment, R¹¹ represents hydrogen, halogen (e.g. fluorine or chlorine), methyl or —CF₃. In a further particular embodiment, R¹¹ represents hydrogen.

In one embodiment of the invention, there is provided a compound of formula (IA), or a pharmaceutically acceptable salt thereof, which is selected from the group consisting of:
N-[(2-chloro-4-fluorophenyl)methyl]-5-oxo-1-[(1R)-1-phenylethyl]-3-pyrrolidinecarboxamide (E1);
N-[(2-Chloro-4-fluorophenyl)methyl]-1-cyclopropyl-5-oxo-3-pyrrolidinecarboxamide (E2);
1-Cyclopropyl-N-[(2,4-dichlorophenyl)methyl]-5-oxo-3-pyrrolidinecarboxamide (E3);
N-{[2-chloro-3-(trifluoromethyl)phenyl]methyl}-1-cyclopropyl-5-oxo-3-pyrrolidinecarboxamide (E4);
N-{[2-chloro-3-(trifluoromethyl)phenyl]methyl}-1-methyl-5-oxo-3-pyrrolidinecarboxamide (E5);
N-[(2,4-dichlorophenyl)methyl]-1-methyl-5-oxo-3-pyrrolidinecarboxamide (E6);
N-[(2,4-dichlorophenyl)methyl]-1-ethyl-5-oxo-3-pyrrolidinecarboxamide (E7);
N-{[2-chloro-3-(trifluoromethyl)phenyl]methyl}-1-ethyl-5-oxo-3-pyrrolidinecarboxamide (E8);
N-{[2-chloro-3-(trifluoromethyl)phenyl]methyl}-1-(2-methylpropyl)-5-oxo-3-pyrrolidinecarboxamide (E9);
N-[(2,4-dichlorophenyl)methyl]-1-(2-methylpropyl)-5-oxo-3-pyrrolidinecarboxamide (E10); and
N-{[2-chloro-3-(trifluoromethyl)phenyl]methyl}-5-oxo-1-(phenylmethyl)-3-pyrrolidinecarboxamide (E11);
   or a pharmaceutically acceptable salt thereof.

In one embodiment of the invention, there is provided a pharmaceutical composition comprising a compound of formula (I), or a pharmaceutically acceptable salt thereof, which is selected from the group consisting of E1-E12.

Antagonists of P2X7 may be useful in preventing, treating, or ameliorating a variety of pain states (e.g. neuropathic pain, chronic inflammatory pain, or visceral pain), inflammation (e.g. rheumatoid arthritis or osteoarthritis) or neurodegenerative diseases, in particular Alzheimer's disease. P2X7 antagonists may constitute useful therapeutic agents in the management of rheumatoid arthritis or inflammatory bowel disease.

Compounds or salts of the present invention which modulate P2X7 receptor function and are capable of antagonizing the effects of ATP at the P2X7 receptor (P2X7 receptor antagonists) may be competitive antagonists, inverse agonists, or negative allosteric modulators of P2X7 receptor function.

Certain compounds of the invention may in some circumstances form acid addition salts thereof. It will be appreciated that for use in medicine compounds of the invention may be used as salts, in which case the salts should be pharmaceutically acceptable. Pharmaceutically acceptable salts include those described by Berge, Bighley and Monkhouse, J. Pharm. Sci., 1977, 66, 1-19.

Basic compounds of the invention may form salts with a pharmaceutically acceptable acid such as an inorganic or organic acid. Such acids include acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid, and the like.

Examples of pharmaceutically acceptable salts include those formed from maleic, fumaric, benzoic, ascorbic, pamoic, succinic, hydrochloric, sulfuric, bismethylenesalicylic, methanesulfonic, ethanedisulfonic, propionic, tartaric, salicylic, citric, gluconic, aspartic, stearic, palmitic, itaconic, glycolic, p-aminobenzoic, glutamic, benzenesulfonic, cyclohexylsulfamic, phosphoric and nitric acids.

The compounds or pharmaceutically acceptable salts of the invention may be prepared in crystalline or non-crystalline form (e.g. in crystalline or amorphous solid form), and, in particular if crystalline, may optionally be solvated, e.g. as the hydrate. This invention includes within its scope solvates (e.g. hydrates) of compounds of formula (I) or (IA) or pharmaceutically acceptable salts thereof, for example stoichiometric solvates (e.g. hydrates); as well as compounds or salts containing variable amounts of solvent (e.g. water).

Certain compounds or salts of the invention are capable of existing in stereoisomeric forms (e.g. diastereomers and enantiomers) and the invention extends to each of these stereoisomeric forms and to mixtures thereof including racemates. The different stereoisomeric forms may be separated one from the other by the usual methods, or any given isomer may be obtained by stereospecific or asymmetric synthesis. The invention also extends to any tautomeric forms and mixtures thereof.

The subject invention also includes isotopically-labelled compounds or salts thereof, which are identical to the compounds of the invention, but for the fact that one or more atoms are replaced by an atom having an atomic mass or mass number different from the atomic mass or mass number most commonly found in nature. Examples of isotopes that can be incorporated into compounds or salts of the invention include isotopes of hydrogen, carbon, nitrogen, oxygen, phosphorous, fluorine, iodine, and chlorine, such as 3H, 11C, 14C, 18F, 123I and 125I.

Compounds of the present invention and pharmaceutically acceptable salts of said compounds that contain the aforementioned isotopes and/or other isotopes of other atoms are within the scope of the present invention. Isotopically-labelled compounds or salts of the present invention, for example those into which radioactive isotopes such as 3H, 14C are incorporated, are potentially useful in drug and/or substrate tissue distribution assays. Tritiated, i.e., 3H, and carbon-14, i.e., 14C, isotopes are for example optionally chosen for their (in some cases) ease of preparation and/or detectability. 11C and 8F isotopes can sometimes be useful in PET (positron emission tomography), and 125I isotopes can sometimes be useful in SPECT (single photon emission computerized tomography). PET and SPECT can sometimes be useful in brain imaging. Further, substitution with heavier isotopes such as deuterium, i.e., 2H, can sometimes afford certain effects resulting from greater metabolic stability, for example increased in vivo half-life or reduced dosage requirements and, hence, may be chosen in some circumstances. Isotopically labelled compounds or salts of this invention are in one embodiment and in some cases prepared by carrying out the procedures disclosed herein, e.g. in the Examples hereinbelow, by substituting an available isotopically labelled reagent for a non-isotopically labelled reagent.

A further particular aspect of the invention provides a compound of formula (I) or (IA) or a pharmaceutically acceptable salt thereof which is not a radioactive isotopically-labelled compound or salt. In a particular embodiment, the compound or salt is not an isotopically-labelled compound or salt.

### Clinical Indications, Pharmaceutical Compositions, and Dosages

It is believed that, as the compounds or pharmaceutically acceptable salts of the present invention modulate P2X7 receptor function and are capable of antagonizing the effects of ATP at the P2X7 receptor (“P2X7 receptor antagonists”); they may be useful in the treatment of pain; such as acute pain, chronic pain, chronic articular pain, musculoskeletal pain, neuropathic pain, inflammatory pain, visceral pain, pain associated with cancer, pain associated with migraine, tension headache or cluster headaches, pain associated with functional bowel disorders, lower back and/or neck pain, pain associated with sprains and/or strains, sympathetically maintained pain; myositis, pain associated with influenza or other viral infections such as the common cold, pain associated with rheumatic fever, pain associated with myocardial ischemia, post operative pain, cancer chemotherapy, headache, toothache, or dysmenorrhea.

The chronic articular pain condition can be rheumatoid arthritis, osteoarthritis, rheumatoid spondylitis (ankylosing spondylitis), gouty arthritis or juvenile arthritis.

The inflammatory pain condition can be rheumatoid arthritis, osteoarthritis, rheumatoid spondylitis (ankylosing spondylitis) or fibromyalgia.

In particular, the compounds of formula (I) or (IA) or pharmaceutically acceptable salts thereof may be useful in the treatment or prevention (treatment or prophylaxis) of pain (e.g. inflammatory pain) in arthritis, such as pain (e.g. inflammatory pain) in rheumatoid arthritis or osteoarthritis.

Pain associated with functional bowel disorders includes non-ulcer dyspepsia, non-cardiac chest pain and irritable bowel syndrome.

The neuropathic pain condition can be: diabetic neuropathy (e.g. painful diabetic neuropathy), sciatica, non-specific lower back pain, trigeminal neuralgia, multiple sclerosis pain, fibromyalgia, HIV-related neuropathy, post-herpetic neuralgia, trigeminal neuralgia, or lumbar radiculopathy; or pain resulting from physical trauma, amputation, phantom limb syndrome, spinal surgery, cancer, toxins or chronic inflammatory conditions. Alternatively, the neuropathic pain condition can be: pain associated with normally non-painful sensations such as “pins and needles” (paraesthesias and/or dysesthesias), increased sensitivity to touch (hyperesthesia), painful sensation following innocuous stimulation (dynamic, static, thermal or cold allodynia), increased sensitivity to noxious stimuli (thermal, cold, or mechanical hyperalgesia), continuing pain sensation after removal of the stimulation (hyperpathia), or an absence of or deficit in selective sensory pathways (hypoalgesia).

The acute pain condition can be post-surgical pain or dysmenorrhea (e.g. primary dysmenorrhea).

Other conditions which could potentially be treated by compounds of the present invention include fever, inflammation, immunological diseases, abnormal platelet function diseases (e.g. occlusive vascular diseases), impotence or erectile dysfunction; bone disease characterised by abnormal bone metabolism or resorbtion; hemodynamic side effects of non-steroidal anti-inflammatory drugs (NSAID's) such as cyclooxygenase-2 (COX-2) inhibitors, cardiovascular diseases; neurodegenerative diseases and neurodegeneration, neurodegeneration following trauma, tinnitus, dependence on a dependence-inducing agent such as opiods (e.g. morphine), CNS (central nervous system) depressants (e.g. ethanol), psychostimulants (e.g. cocaine) or nicotine; complications of Type I diabetes, kidney dysfunction, liver dysfunction (e.g. hepatitis, cirrhosis), gastrointestinal dysfunction (e.g. diarrhoea), colon cancer, overactive bladder, and urge incontinence. Depression and alcoholism could potentially also be treated by compounds or salts of the present invention.

Inflammation and the inflammatory conditions associated with said inflammation include arthritis (in particular rheumatoid arthritis or osteoarthritis), skin conditions (e.g. sunburn, burns, eczema, dermatitis, allergic dermatitis, psoriasis), meningitis, ophthalmic diseases such as glaucoma, retinitis, retinopathies, uveitis and of acute injury to the eye tissue (e.g. conjunctivitis), inflammatory lung disorders (e.g. asthma, allergic rhinitis, respiratory distress syndrome, pigeon fancier's disease, farmer's lung, chronic obstructive pulmonary disease (COPD, which includes bronchitis and/or emphysema), or airways hyperresponsiveness); gastrointestinal tract disorders (e.g. aphthous ulcer, Crohn's disease, atopic gastritis, gastritis varialoforme, ulcerative colitis, coeliac disease, regional ileitis, irritable bowel syndrome, inflammatory bowel disease, or gastrointestinal reflux disease); organ transplantation and other conditions with an inflammatory component such as vascular disease, migraine, periarteritis nodosa, thyroiditis, aplastic anaemia, Hodgkin's disease, sclerodoma, myaesthenia gravis, multiple sclerosis, sorcoidosis, nephrotic syndrome, Bechet's syndrome, gingivitis, myocardial ischemia, pyrexia, systemic lupus erythematosus, polymyositis, tendinitis, bursitis, and Sjogren's syndrome. Inflammation or an inflammatory condition associated with said inflammation can in particular be arthritis (e.g. rheumatoid arthritis or osteoarthritis).

Immunological diseases include autoimmune diseases, immunological deficiency diseases or organ transplantation.

Bone diseases characterised by abnormal bone metabolism or resorbtion include osteoporosis (especially postmenopausal osteoporosis), hyper-calcemia, hyperparathyroidism, Paget's bone diseases, osteolysis, hypercalcemia of malignancy with or without bone metastases, rheumatoid arthritis, periodontitis, osteoarthritis, ostealgia, osteopenia, cancer cacchexia, calculosis, lithiasis (especially urolithiasis), solid carcinoma, gout and ankylosing spondylitis, tendinitis and bursitis.

A bone disease characterised by abnormal bone metabolism or resorbtion may particular be rheumatoid arthritis or osteoarthritis, for potential treatment by compounds or pharmaceutically acceptable salts of the present invention.

Cardiovascular diseases include hypertension or myocardiac ischemia; atherosclerosis; functional or organic venous insufficiency; varicose therapy; haemorrhoids; and shock states associated with a marked drop in arterial pressure (e.g. septic shock).

Neurodegenerative diseases include dementia, particularly degenerative dementia (such as senile dementia, dementia with Lewy bodies, Alzheimer's disease, Pick's disease, Huntingdon's chorea, Parkinson's disease and Creutzfeldt-Jakob disease, Amyotrophic Lateral Sclerosis (ALS) or motor neuron disease; in particular Alzheimer's disease); vascular dementia (including multi-infarct dementia); as well as dementia associated with intracranial space occupying lesions; trauma; infections and related conditions (including HIV infection, meningitis and shingles); metabolism; toxins; anoxia and vitamin deficiency; and mild cognitive impairment e.g. associated with ageing, particularly age associated memory impairment.

The neurodegenerative disease, e.g. to be treated by the compound of formula (I) or (IA) or salt thereof, can for example be degenerative dementia (in particular Alzheimer's disease), vascular dementia (in particular multi-infarct dementia), or mild cognitive impairment (MCI) e.g. MCI associated with ageing such as age associated memory impairment.

The compounds or salts of the invention may also be useful for neuroprotection and in the treatment of neurodegeneration following trauma such as stroke, cardiac arrest, pulmonary bypass, traumatic brain injury, spinal cord injury or the like.

The compounds or salts of the present invention may also be useful in the treatment of malignant cell growth and/or metastasis, and myoblastic leukaemia.

Complications of Type 1 diabetes include diabetic microangiopathy, diabetic retinopathy, diabetic nephropathy, macular degeneration, glaucoma, nephrotic syndrome, aplastic anaemia, uveitis, Kawasaki disease and sarcoidosis.

Kidney dysfunction includes nephritis, glomerulonephritis, particularly mesangial proliferative glomerulonephritis and nephritic syndrome.

It is to be understood that reference to treatment includes both treatment of established symptoms and prophylactic treatment, unless explicitly stated otherwise.

According to a further aspect of the invention, we therefore provide a compound of formula (I) or (IA) or a pharmaceutically acceptable salt thereof for use in human or veterinary medicine; and/or for use in therapy.

According to another aspect of the invention, we provide a compound of formula (I) or (IA) or a pharmaceutically acceptable salt thereof for use in the treatment or prevention (e.g. treatment) of a condition which is mediated by P2X7 receptors, for example a condition or disease disclosed herein (in particular pain, inflammation such as rheumatoid arthritis or osteoarthritis, or a neurodegenerative disease; more particularly pain such as inflammatory pain, neuropathic pain or visceral pain, or rheumatoid arthritis or osteoarthritis); e.g. in a mammal such as a human or rodent e.g. human or rat e.g. human.

According to a further aspect of the invention, we provide a method of treating a human or animal (e.g. rodent e.g. rat) subject, for example a human subject, suffering from a condition which is mediated by P2X7 receptors, for example a condition or disease disclosed herein (in particular pain, inflammation such as rheumatoid arthritis or osteoarthritis, or a neurodegenerative disease; more particularly pain such as inflammatory pain, neuropathic pain or visceral pain, or rheumatoid arthritis or osteoarthritis), which comprises administering to said subject an effective amount of a compound of formula (I) or (IA) or a pharmaceutically acceptable salt thereof.

According to a further aspect of the invention we provide a method of treating a human or animal (e.g. rodent e.g. rat) subject, for example a human subject, suffering from pain, inflammation (e.g. rheumatoid arthritis or osteoarthritis), or a neurodegenerative disease (more particularly pain such as inflammatory pain, neuropathic pain or visceral pain, or rheumatoid arthritis or osteoarthritis), which method comprises administering to said subject an effective amount of a compound of formula (I) or (IA) or a pharmaceutically acceptable salt thereof.

According to a yet further aspect of the invention we provide a method of treating a human or animal (e.g. rodent e.g. rat) subject, for example a human subject, suffering from inflammatory pain, neuropathic pain or visceral pain (e.g. pain, such as inflammatory pain, in arthritis (e.g. rheumatoid arthritis or osteoarthritis)) which method comprises administering to said subject an effective amount of a compound of formula (I) or (IA) or a pharmaceutically acceptable salt thereof.

According to a further aspect of the invention we provide a method of treating a subject, for example a human subject, suffering from Alzheimer's disease which method comprises administering to said subject an effective amount of a compound of formula (I) or (IA) or a pharmaceutically acceptable salt thereof.

According to another aspect of the invention, we provide the use of a compound of formula (I) or (IA) or a pharmaceutically acceptable salt thereof for the manufacture of a medicament for the treatment or prevention (e.g. treatment) of a condition which is mediated by the action of P2X7 receptors, for example a condition or disease disclosed herein (in particular pain, inflammation such as rheumatoid arthritis or osteoarthritis, or a neurodegenerative disease; more particularly pain such as inflammatory pain, neuropathic pain or visceral pain); e.g. in a mammal such as a human or rodent e.g. human or rat e.g. human.

According to another aspect of the invention we provide the use of a compound of formula (I) or (IA) or a pharmaceutically acceptable salt thereof for the manufacture of a medicament for the treatment or prevention (e.g. treatment) of pain (e.g. inflammatory pain, neuropathic pain or visceral pain), inflammation (e.g. rheumatoid arthritis or osteoarthritis), or a neurodegenerative disease (more particularly: pain such as inflammatory pain, neuropathic pain or visceral pain, or rheumatoid arthritis or osteoarthritis); e.g. in a mammal such as a human or rodent e.g. human or rat e.g. human.

According to another aspect of the invention we provide the use of a compound of formula (I) or (IA) or a pharmaceutically acceptable salt thereof for the manufacture of a medicament for the treatment or prevention (e.g. treatment) of inflammatory pain, neuropathic pain or visceral pain (in particular inflammatory pain or neuropathic pain; such as inflammatory pain in arthritis such as rheumatoid arthritis or osteoarthritis); e.g. in a mammal such as a human or rodent e.g. human or rat e.g. human.

In one aspect of the invention we provide the use of a compound of formula (I) or (IA) or a pharmaceutically acceptable salt thereof for the manufacture of a medicament for the treatment or prevention (e.g. treatment) of Alzheimer's disease; e.g. in a mammal such as a human or rodent e.g. human or rat e.g. human.

In order to use a compound of the invention or a pharmaceutically acceptable salt thereof for the treatment of humans and/or other mammals it can optionally be formulated in accordance with pharmaceutical practice as a pharmaceutical composition. Therefore in another aspect of the invention there is provided a pharmaceutical composition comprising a compound of formula (I) or (IA), or a pharmaceutically acceptable salt thereof, adapted for use in human or veterinary medicine.

In order to use a compound of formula (I) or (IA) or a pharmaceutically acceptable salt thereof in therapy, it will normally be formulated into a pharmaceutical composition in accordance with pharmaceutical practice. The present invention also provides a pharmaceutical composition, which comprises a compound of formula (I) or (IA), or a pharmaceutically acceptable salt thereof, and usually a pharmaceutically acceptable carrier or excipient.

The pharmaceutical composition may be for use in a method of treatment or in a use or in a treatment or prevention, as described herein.

A pharmaceutical composition of the invention, which may be prepared by admixture, for example at ambient temperature and/or atmospheric pressure, is usually adapted for oral, parenteral or rectal administration. As such, the pharmaceutical composition may be in the form of a tablet, a capsule, a oral liquid preparation, a powder, a granule, a lozenge, a reconstitutable powder, an injectable or infusable solution or suspension, or a suppository.

An orally administrable pharmaceutical composition is generally preferred.

Tablets and capsules for oral administration may be in unit dose form, and may contain one or more excipients, such as a binding agent (e.g. hydroxypropylmethylcellulose or povidone), a filler (e.g. lactose and/or microcrystalline cellulose), a lubricant e.g. a tabletting lubricant (e.g. magnesium stearate or calcium stearate), a disintegrant (e.g. a tablet disintegrant such as sodium starch glycolate or croscarmellose sodium), and/or an acceptable wetting agent. The tablets may be coated e.g. according to methods known in pharmaceutical practice.

Oral liquid preparations may be in the form of, for example, aqueous or oily suspension, solutions, emulsions, syrups or elixirs, or may be in the form of a dry product for reconstitution with water or other suitable vehicle before use. Such liquid preparations may contain additive(s) such as a suspending agent(s), an emulsifying agent(s), a non-aqueous vehicle(s) (such as an edible oil), and/or a preservative(s), and/or, if desired, a flavouring(s) or colourant(s).

For parenteral administration, fluid unit dosage forms are typically prepared utilising a compound of the invention or pharmaceutically acceptable salt thereof and a sterile vehicle. In one embodiment, the compound or salt, depending on the vehicle and concentration used, is either suspended or dissolved in the vehicle. In preparing solutions, the compound or salt can e.g. be dissolved for injection and filter sterilised before filling into a suitable vial or ampoule and sealing. In one embodiment, an adjuvant(s) such as a local anaesthetic, a preservative(s) and/or a buffering agent(s) is or are dissolved in the vehicle. To enhance the stability, the composition can for example be frozen after filling into the vial and the water removed under vacuum. Parenteral suspensions are typically prepared in substantially the same manner, except that the compound or salt is typically suspended in the vehicle instead of being dissolved, and sterilization is not usually accomplished by filtration. The compound or salt can be sterilised, e.g. by exposure to ethylene oxide, before suspension in a sterile vehicle. In one embodiment, a surfactant or wetting agent is included in the composition, e.g. to facilitate uniform distribution of the compound or salt of the invention.

In one embodiment, the composition contains from 0.1% to 99% (by weight of the composition), in particular from 0.1 to 60% or 1 to 60% or 10 to 60% by weight, of the active material (the compound or pharmaceutically acceptable salt of the invention), e.g. depending on the method of administration. The carrier(s) and/or excipient(s) contained in the composition can for example be present in from 1% to 99.9%, e.g. from 10% to 99%, by weight of the composition; and/or in an amount of from 20 mg to 2000 mg such as 50 mg to 1000 mg per unit dose of the composition.

The dose of the compound or pharmaceutically acceptable salt thereof, e.g. for use in the treatment or prevention (e.g. treatment) of the hereinmentioned disorders/diseases/conditions, may vary in the usual way with the seriousness of the disorders, the weight of the sufferer, and/or other similar factors. However, as a general guide, in one embodiment a unit dose of 0.05 to 2000 mg or 0.05 to 1000 mg, for example 0.05 to 200 mg, such as 20 to 40 mg, of the compound or pharmaceutically acceptable salt of the invention (measured as the compound), may be used, e.g. in a pharmaceutical composition. In one embodiment, such a unit dose is for administration once a day e.g. to a mammal such as a human; alternatively such a unit dose may be for administration more than once (e.g. twice or three times) a day e.g. to a mammal such as a human. Such therapy may extend for a number of days, weeks, months or years.

### Combinations

Compounds of formula (I) or (IA) or pharmaceutically acceptable salts thereof may be used in combination with other (further) therapeutic agents, for example medicaments claimed to be useful in the treatment or prevention (e.g. treatment) of the above mentioned disorders.

Examples of other such further therapeutic agents may include a β2-agonist (also known as β2 adrenoceptor agonists; e.g. formoterol) and/or a corticosteroid (e.g. budesonide, fluticasone (e.g. as propionate or furoate esters), mometasone (e.g. as furoate), beclomethasone (e.g. as 17-propionate or 17,21-dipropionate esters), ciclesonide, triamcinolone (e.g. as acetonide), flunisolide, rofleponide and butixocort (e.g. as propionate ester), e.g. for the treatment of respiratory disorders (such as asthma or chronic obstructive pulmonary disease (COPD)), e.g. as described in WO 2007/008155 and WO 2007/008157.

A further therapeutic agent may include a 3-hydroxy-3-methylglutaryl coenzyme A (HMG CoA) reductase inhibitor (e.g. atorvastatin, fluvastatin, lovastatin, pravastatin, rosuvastatin, or simvastatin) (e.g. for oral administration), e.g. for the treatment of cardiovascular disorders (such as atherosclerosis), e.g. as described in WO 2006/083214.

A further therapeutic agent may in particular include a non-steroid anti-inflammatory drug (NSAID; e.g. ibuprofen, naproxen, aspirin, celecoxib, diclofenac, etodolac, fenoprofen, indomethacin, ketoprofen, ketoralac, oxaprozin, nabumetone, sulindac, tolmetin, rofecoxib, valdecoxib, lumaricoxib, meloxicam, etoricoxiband or parecoxib; or e.g. paracetamol, loxoprofen or aceclofenac; in particular celecoxib, paracetamol, ibuprofen or diclofenac) (e.g. for oral administration), e.g. for the treatment of an inflammatory disease or disorder (such as rheumatoid arthritis or osteoarthritis, and/or inflammatory pain), e.g. as described in WO 2005/025571. Celecoxib (a COX-2 inhibitor) can for example be administered orally at a dosage regimen of 100 mg or 200 mg (measured as the free base) once or twice daily.

A further therapeutic agent may in particular include a tumour necrosis factor α (TNFα) inhibitor (e.g. etanercept or an anti-TNFα antibody such as infliximab and adalimumab) (e.g. for parenteral administration such as subcutaneous or intravenous administration), e.g. for the treatment of an inflammatory disease or disorder (such as rheumatoid arthritis or osteoarthritis), e.g. as described in WO 2004/105798.

A further therapeutic agent may in particular include an anti-CD20 monoclonal antibody (e.g. for parenteral such as intravenous administration), such as ofatumumab (HuMax-CD20™, developed in part by Genmab AS) (e.g. ofatumumab for intravenous administration), rituximab, PRO70769, AME-133 (Applied Molecular Evolution), or hA20 (Immunomedics, Inc.); in particular ofatumumab or rituximab. This further therapeutic agent can e.g. be for the treatment of an inflammatory disease or disorder (such as rheumatoid arthritis or osteoarthritis, and/or inflammatory pain).

A further therapeutic agent may include 2-hydroxy-5-[[4-[(2-pyridinylamino) sulfonyl]phenyl]azo]benzoic acid (sulfasalazine), e.g. for the treatment of an inflammatory disease or disorder (such as rheumatoid arthritis), e.g. as described in WO 2004/105797.

A further therapeutic agent may in particular include N-[4-[[(2,4-diamino-6-pteridinyl)methyl]methylamino]benzoyl]-L-glutamic acid (methotrexate), e.g. for oral administration and/or e.g. for the treatment of an inflammatory disease or disorder (such as rheumatoid arthritis), e.g. as described in WO 2004/105796. For the treatment of rheumatoid arthritis, methotrexate can be administered to the human at a dosage regimen of 7.5 mg orally once weekly, or using divided oral doses of 2.5 mg at 12 hour intervals for 3 doses (7.5 mg total) as a course once weekly; the schedule can optionally be adjusted gradually to achieve an optimal response, but typically does not exceed a total weekly oral dose of 20 mg of methotrexate; once a response has been achieved, the methotrexate dose is typically reduced to the lowest possible effective dose.

A further therapeutic agent may include an inhibitor of pro TNFα convertase enzyme (TACE), e.g. for the treatment of an inflammatory disease or disorder (such as rheumatoid arthritis), e.g. as described in WO 2004/073704.

A further therapeutic agent may include:

a) sulfasalazine;
b) a statin (e.g. for oral administration), such as atorvastatin, lovastatin, pravastatin, simvastatin, fluvastatin, cerivastatin, crilvastatin, dalvastatin, rosuvastatin, tenivastatin, fluindostatin, velostatin, dalvastatin, nisvastatin, bervastatin, pitavastatin, rivastatin, glenvastatin, eptastatin, tenivastatin, flurastatin, rosuvastatin or itavastatin;
c) a glucocorticoid agent (e.g. for oral or skin-topical administration), such as dexamethasone, methylprednisolone, prednisolone, prednisone and hydrocortisone;
d) an inhibitor of p38 kinase (e.g. for oral administration);
e) an anti-IL-6-receptor antibody, e.g. an anti-IL-6-receptor monoclonal antibody (e.g. for parenteral such as intravenous administration);
f) anakinra;
g) an anti-IL-1 (e.g. IL-1β) monoclonal antibody (e.g. for parenteral such as intravenous administration);
h) an inhibitor of JAK3 protein tyrosine kinase;
i) an anti-macrophage colony stimulation factor (M-CSF) monoclonal antibody; or
j) an anti-CD20 monoclonal antibody (e.g. for parenteral such as intravenous administration), such as rituximab, ofatumumab (HuMax-CD20™, developed in part by Genmab AS) (e.g. ofatumumab for intravenous administration), PRO70769, AME-133 (Applied Molecular Evolution), or hA20 (Immunomedics, Inc.); in particular rituximab or ofatumumab;
e.g. for the treatment of an IL-1 (e.g. IL-1β) mediated disease (such as rheumatoid arthritis or osteoarthritis, and/or inflammatory or neuropathic pain; in particular rheumatoid arthritis), e.g. as described in WO 2006/003517.

In particular, the further therapeutic agent or agents can be a therapeutic agent or agents capable of treating inflammatory pain, such as paracetamol and/or an opioid (such as morphine, fentanyl, oxycodone, tramadol, hydrocodone, hydromorphone, oxymorphone, methadone or buprenorphine; in particular morphine, fentanyl, oxycodone, or tramadol). This/these therapeutic agent(s), and/or the combination comprising this/these therapeutic agent(s), can be for the treatment of inflammatory pain, e.g. in a mammal such as a human. For example, paracetamol can be administered at a human oral dosage regimen of 500 mg to 1000 mg (e.g. 500 mg, 650 mg or 1000 mg, in particular 650 mg) of paracetamol (measured as the free base/free compound), administered two, three or four times daily.

In a particular embodiment of the invention, the further therapeutic agent or agents can be a therapeutic agent or agents capable of treating neuropathic pain, such as:
an opioid (such as morphine, fentanyl, oxycodone, tramadol, hydrocodone, hydromorphone, oxymorphone, methadone or buprenorphine; in particular morphine, fentanyl, oxycodone, or tramadol, most particularly morphine),
a monoamine reuptake inhibitor (such as duloxetine or amytriptyline),
pregabalin,
gabapentin,
gabapentin enacarbil (XP13512), and/or
carbamazepine.

This/these therapeutic agent(s), and/or the combination comprising this/these therapeutic agent(s), can be for the treatment of neuropathic pain, e.g. in a mammal such as a human.

For example, pregabalin can be administered orally e.g. for neuropathic pain; e.g. at a human oral dosage regimen of 150 mg to 600 mg total pregabalin per day (measured as the free base), split between two to three doses per day. For example, for postherpetic neuralgia (a neuropathic pain condition), pregabalin can be administered at a starting oral dosage regimen of 150 mg total pregabalin per day (split between 2 or 3 doses per day), escalating (e.g. in about one week) to an oral dosage regimen of 300 mg pregabalin total per day, and optionally escalating up to a maximum oral dosage regimen of 600 mg total pregabalin per day. For painful diabetic neuropathy (another neuropathic pain condition), an oral dosage regimen of 150 mg to 300 mg total pregabalin per day can be administered. For fibromyalgia, an oral dosage regimen of 150 mg to 450 mg (e.g. 300 or 450 mg) total pregabalin per day can be administered. Pregabalin can e.g. be administered separately from the compound or salt of the invention.

For example, gabapentin can be administered orally, e.g. for neuropathic pain. Oral dosage units can e.g. contain 100 mg, 300 mg, 400 mg, 600 mg or 800 mg of gabapentin (measured as the free base/acid). The gabapentin dosage regimen for neuropathic pain can e.g. be from 300 mg once, twice or three times per day up to a total dose of 3600 mg/day. Some gradual up-titration of the dosage regimen is usually performed. For example, for peripheral neuropathic pain in adults, gabapentin therapy can be initiated by titrating the dose thus: day 1=300 mg of gabapentin (measured as the free base/acid) once a day, day 2=300 mg two times a day, and day 3=300 mg three times a day; alternatively the starting dose can be 900 mg/day of gabapentin (measured as the free base/acid), administered as three equally divided doses. Thereafter, e.g. based on individual patient response and tolerability, the dose can be further increased, typically in 300 mg/day increments every 2-3 days, up to a maximum total dose of 3600 mg/day of gabapentin (measured as the free base/acid). Slower titration of gabapentin dosage may be appropriate for individual patients. The minimum time to reach a total dose of 1800 mg/day is typically one week, to reach 2400 mg/day is typically a total of 2 weeks, and to reach 3600 mg/day is typically a total of 3 weeks. Gabapentin can e.g. be administered separately from the compound or salt of the invention.

For example, gabapentin enacarbil (XP13512, (±)-1-([(α-isobutanoyloxyethoxy)carbonyl]aminomethyl)-1-cyclohexane acetic acid, which is a prodrug of gabapentin) can be administered orally, e.g. to a human, e.g. separately from the compound or salt of the invention. In one embodiment, gabapentin enacarbil (XP13512) is for example administered orally, e.g. to a human such as a human adult, e.g. at a total daily dose having an equivalent molar quantity of gabapentin enacarbil as the molar quantity present in 900 mg/day to 3600 mg/day of gabapentin (see e.g. page 81 lines 24-32 of WO 02/100347). A 600 mg dose of gabapentin enacarbil (measured as the free acid) contains the molar equivalent of 312 mg of gabapentin. See also K. C. Cundy et al., “Clinical Pharmacokinetics of XP13512, a Novel Transported Prodrug of Gabapentin”, J. Clin. Pharmacol., 2008, e-publication 30 Sep. 2008, incorporated herein by reference, and the Materials and Methods—Formulation and Study Designs sections therein, for examples of some oral doses, dosage regimens and formulations of XP13512 used in human pharmacokinetic studies.

In a particular embodiment of the invention, when the further therapeutic agent includes an opioid (such as morphine, fentanyl, oxycodone, tramadol, hydrocodone, hydromorphone, oxymorphone, methadone or buprenorphine; in particular morphine, fentanyl, oxycodone, or tramadol), then the opioid and/or the combination comprising the opioid is for the treatment of pain, in particular inflammatory or neuropathic pain, e.g. in a mammal such as a human.

When the compounds are used in combination with other therapeutic agents, the compounds may be administered either sequentially or simultaneously by any convenient route.

The invention thus provides, in a further aspect, a combination comprising a compound of the invention or a pharmaceutically acceptable salt thereof together with a further therapeutic agent or agents (e.g. as defined herein).

The individual components of the combination of the invention (i.e. the compound of formula (I) or (IA) or the salt thereof, and the further therapeutic agent or agents) may be present as separate pharmaceutical formulations/compositions, or may be present as a combined pharmaceutical formulation/composition (e.g. may be together in a single combined oral dosage form, e.g. a single combined tablet or capsule). The individual components of this combination can for example be administered either sequentially in separate pharmaceutical formulations/compositions (e.g. oral), or simultaneously in separate or combined pharmaceutical formulation(s)/composition(s) (e.g. oral); in a particular embodiment they are administered sequentially in separate pharmaceutical formulations/compositions (e.g. oral).

The combinations referred to herein may optionally be presented for use in the form of a pharmaceutical formulation and thus pharmaceutical formulations comprising a combination as defined herein together with a pharmaceutically acceptable carrier or excipient comprise a further aspect of the invention. The individual components of such combinations may be administered either sequentially or simultaneously in separate or combined pharmaceutical formulations.

When a compound of the invention or a pharmaceutically acceptable salt thereof is used in combination with a second therapeutic agent active against the same disease state the dose of each compound may differ from that when the compound is used alone.

The following Descriptions and Examples illustrate the preparation of compounds of the invention but are not intended to be limiting.

### EXAMPLES

Abbreviations, some of which may be used herein, include the following:
Boc tert-butyl oxy carbonyl
DMSO dimethyl sulfoxide
DCM dichloromethane
DMF N,N-dimethylformamide
DIPEA N,N-diisopropylethyl amine (ⁱPr₂NEt)
EDC 1-(3-dimethylaminopropyl)-3-ethylcarbodiimide hydrochloride
EtOAc ethyl acetate
Et₂O diethyl ether
EtOH ethanol
HEPES 4-(2-hydroxyethyl)-1-piperazine-1-ethanesulfonic acid

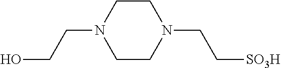

HOBT 1-hydroxybenzotriazole
IPA isopropanol (isopropyl alcohol)
MeCN acetonitrile
MeOH methanol
THF tetrahydrofuran
TFA trifluoroacetic acid
eq equivalents
HPLC high performance liquid chromatography
h hours
min minutes
LCMS or LC/MS liquid chromatography/mass spectroscopy
MDAP mass directed automated (preparative) HPLC
NMR nuclear magnetic resonance
TLC thin layer chromatography
RT room temperature (ambient temperature); this is usually in the range of about 18 to about 25° C., or a sub-range within this range, except as disclosed herein.

### Example 1

### N-[(2-chloro-4-fluorophenyl)methyl]-5-oxo-1-[(1R)-1-phenylethyl]-3-pyrrolidinecarboxamide (E1)

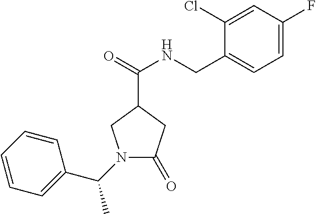

(3S)-5-Oxo-1-[(1R)-1-phenylethyl]-3-pyrrolidinecarboxylic acid (50 mg, 0.21 mmol), 2-chloro-4-fluorobenzylamine (38 mg, 0.24 mmol), 1-(3-dimethylaminopropyl)-3-ethylcarbodiimide hydrochloride (EDC) (45 mg, 0.24 mmol), 1-hydroxybenzotriazole (HOBT) (32 mg, 0.24 mmol) and N-ethyl morpholine (30 uL, 0.24 mmol) were combined in DCM:DMF (dichloromethane:dimethylformamide) (3:2) (5 ml) at 0° C. and stirred overnight after allowing the mixture to reach room temperature. The mixture was diluted with 2M HCl and dichloromethane and the dichloromethane layer was separated and washed with saturated sodium bicarbonate solution. The biphasic system was filtered through a phase separator and evaporation of the dichloromethane gave the crude product. The crude product was purified by MDAP to give the pure product as a clear oil. Freeze drying afforded the title product, N-[(2-chloro-4-fluorophenyl)methyl]-5-oxo-1-[(1R)-1-phenylethyl]-3-pyrrolidinecarboxamide as a white solid. LC/MS [M+H]⁺375, retention time=2.76 min.

### Example 2

### N-[(2-Chloro-4-fluorophenyl)methyl]-1-cyclopropyl-5-oxo-3-pyrrolidinecarboxamide (E2)

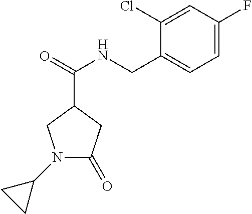

1-Cyclopropyl-5-oxo-3-pyrrolidinecarboxylic acid (100 mg, 0.59 mmol) was dissolved in dichloromethane (DCM). 2-Chloro-4-fluorobenzylamine (103 mg, 0.65 mmol), 1-hydroxybenzotriazole (HOBT) (88 mg, 0.65 mmol), N-ethyl morpholine (83 uL, 0.65 mmol) and 1-(3-dimethylaminopropyl)-3-ethylcarbodiimide hydrochloride (EDC) (124 mg, 0.65 mmol) were added under argon at room temperature and stirred for 24 hours. The mixture was diluted with 2M HCl and dichloromethane and the dichloromethane layer was separated and washed with saturated sodium bicarbonate solution. The biphasic system was filtered through a phase separator and evaporation of the dichloromethane gave the crude product. The crude product was purified by MDAP to give the title product, N-[(2-Chloro-4-fluorophenyl)methyl]-1-cyclopropyl-5-oxo-3-pyrrolidinecarboxamide, as a clear oil, 65 mg. LC/MS [M+H]⁺ 311, retention time=2.12 min.

### Example 3

### 1-Cyclopropyl-N-[(2,4-dichlorophenyl)methyl]-5-oxo-3-pyrrolidinecarboxamide (E3)

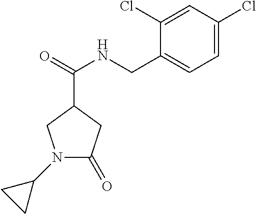

1-Cyclopropyl-5-oxo-3-pyrrolidinecarboxylic acid (169 mg, 1 mmol), 1-hydroxybenzotriazole (HOBT) (306 mg, 2 mmol) and 1-(3-dimethylaminopropyl)-3-ethylcarbodiimide hydrochloride (EDC) (383 mg, 2 mmol) were stirred at room temperature for 30 minutes in dichloromethane (DCM) (5 ml). 2,4-Dichlorobenzylamine (199 uL, 1.5 mol) was added and the solution stirred at room temperature overnight. The reaction mixture was concentrated and the residue partitioned between ethyl acetate and water. The water was back extracted with ethyl acetate and the organic phases combined. The combined ethyl acetate extracts were washed with 3N citric acid solution, water (×2), saturated sodium bicarbonate solution, water (×2), brine, dried over anhydrous magnesium sulphate and concentrated in vacuo to afford a crude solid. The crude product was purified by MDAP to afford the title product, 1-Cyclopropyl-N-[(2,4-dichlorophenyl)methyl]-5-oxo-3-pyrrolidinecarboxamide (156 mg).

LC/MS [M+H]⁺ 327/329/331, retention time=2.37 min.

### Examples 4-12

In a manner analogous to that described for Example 3 above, the compounds tabulated below (Table 1) were prepared by substituting the appropriate acid for the 1-cyclopropyl-5-oxo-3-pyrrolidinecarboxylic acid and the appropriate benzylic amine for the 1-[2-chloro-3-(trifluoromethyl)phenyl]methanamine used in the above procedure. All of the acids and benzylamines used to prepare the compounds listed in Table 1 are available from commercial sources or can be prepared using methods described in the chemical literature.

TABLE 1

| | | | Retention |
|---|---|---|---|
| Example | | | time |
| no. | Chemical name | [M + H]⁺ | (mins) |
| E4 | 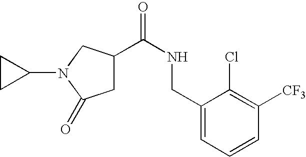 | 361/363 | 2.40 |
| | N-{[2-chloro-3- (trifluoromethyl)phenyl]methyl}-1- cyclopropyl-5-oxo-3-pyrrolidinecarboxamide | | |
| E5 | 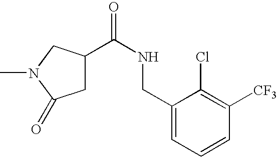 | 335/337 | 2.26 |
| | N-{[2-chloro-3- (trifluoromethyl)phenyl]methyl}-1-methyl-5- oxo-3-pyrrolidinecarboxamide | | |
| E6 | 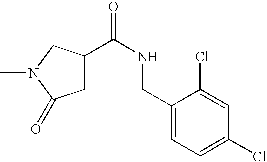 | 301/303/305 | 2.13 |
| | N-[(2,4-dichlorophenyl)methyl]-1-methyl-5- oxo-3-pyrrolidinecarboxamide | | |
| E7 | 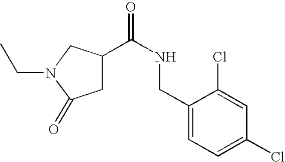 | 315/317/319 | 2.28 |
| | N-[(2,4-dichlorophenyl)methyl]-1-ethyl-5- oxo-3-pyrrolidinecarboxamide | | |
| E8 | 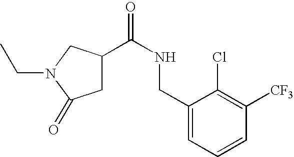 | 349/351 | 2.37 |
| | N-{[2-chloro-3- (trifluoromethyl)phenyl]methyl}-1-ethyl-5- oxo-3-pyrrolidinecarboxamide | | |
| E9 | 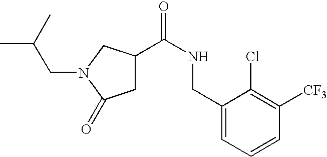 | 377/379 | 2.67 |
| | N-{[2-chloro-3- (trifluoromethyl)phenyl]methyl}-1-(2- methylpropyl)-5-oxo-3- pyrrolidinecarboxamide | | |
| E10 | 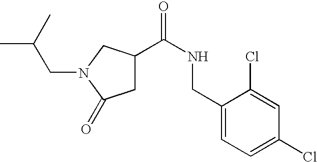 | 343/345/347 | 2.60 |
| | N-[(2,4-dichlorophenyl)methyl]-1-(2- methylpropyl)-5-oxo-3- pyrrolidinecarboxamide | | |
| E11 | 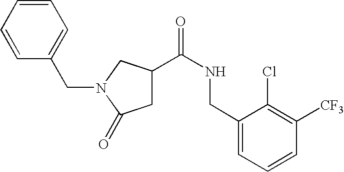 | 411/413 | 2.78 |
| | N-{[2-chloro-3- (trifluoromethyl)phenyl]methyl}-5-oxo-1- (phenylmethyl)-3-pyrrolidinecarboxamide | | |
| E12 | 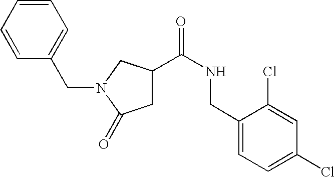 | 377/379/381 | 2.71 |
| | N-[(2,4-dichlorophenyl)methyl]-5-oxo-1- (phenylmethyl)-3-pyrrolidinecarboxamide | | |

### Example 5A

### N-{[2-chloro-3-(trifluoromethyl)phenyl]methyl}-1-methyl-5-oxo-3-pyrrolidinecarboxamide—separation of isomers

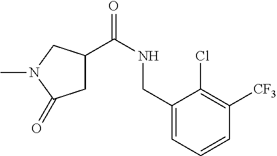

N-{[2-Chloro-3-(trifluoromethyl)phenyl]methyl}-1-methyl-5-oxo-3-pyrrolidinecarboxamide (E5), substantially as prepared in the method given in Example 5 above, was dissolved in solvent(s) and passed through a column with a chiral stationary phase to separate two stereoisomers, each of whose absolute stereochemistry was not known. The two stereoisomers were isolated.

It is assumed, but not confirmed, that one stereoisomer will be:

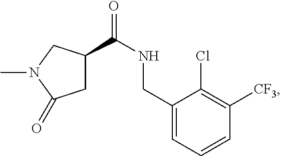

and that the other stereoisomer will be:

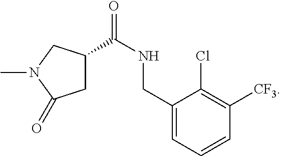

### Mass-Directed Automated HPLC (MDAP)

Where indicated in the above examples, purification by mass-directed automated HPLC was carried out using the following apparatus and conditions:

### Hardware

### Waters 2525 Binary Gradient Module

### Waters 515 Makeup Pump

### Waters Pump Control Module

### Waters 2767 Inject Collect

### Waters Column Fluidics Manager

### Waters 2996 Photodiode Array Detector

### Waters ZQ Mass Spectrometer

Gilson 202 fraction collector
Gilson Aspec waste collector

### Software

Waters MassLynx version 4 SP2

### Column

The columns used are Waters Atlantis, the dimensions of which are 19 mm×100 mm (small scale) and 30 mm×100 mm (large scale). The stationary phase particle size is 5 μm.

### Solvents

A: Aqueous solvent=Water+0.1% Formic Acid
B: Organic solvent=Acetonitrile+0.1% Formic Acid
Make up solvent=Methanol:Water 80:20
Needle rinse solvent=Methanol

### Methods

There are five methods used depending on the analytical retention time of the compound of interest. They have a 13.5-minute runtime, which comprises a 10-minute gradient followed by a 3.5 minute column flush and re-equilibration step.

### Large/Small Scale 1.0-1.5=5-30% B

### Large/Small Scale 1.5-2.2=15-55% B

### Large/Small Scale 2.2-2.9=30-85% B

### Large/Small Scale 2.9-3.6=50-99% B

Large/Small Scale 3.6-5.0=80-99% B (in 6 minutes followed by 7.5 minutes flush and re-equilibration)

### Flow Rate

All of the above methods have a flow rate of either 20 mls/min (Small Scale) or 40 mls/min (Large Scale).

### Liquid Chromatography/Mass Spectrometry

Analysis of the above Examples by Liquid Chromatography/Mass Spectrometry (LC/MS) was carried out using the following apparatus and conditions:

### Hardware

### Agilent 1100 Gradient Pump

### Agilent 1100 Autosampler

### Agilent 1100 DAD Detector

### Agilent 1100 Degasser

### Agilent 1100 Oven

### Agilent 1100 Controller

### Waters ZQ Mass Spectrometer

### Sedere Sedex 85

### Software

Waters MassLynx version 4.0 SP2

### Column

The column used is a Waters Atlantis, the dimensions of which are 4.6 mm×50 mm. The stationary phase particle size is 3 μm.

### Solvents

A: Aqueous solvent=Water+0.05% Formic Acid
B: Organic solvent=Acetonitrile+0.05% Formic Acid

### Method

The generic method used has a 5 minute runtime.

| Time/min | % B |
|---|---|
| 0 | 3 |
| 0.1 | 3 |
| 4 | 97 |
| 4.8 | 97 |
| 4.9 | 3 |
| 5.0 | 3 |

The above method has a flow rate of 3 ml/mins.

The injection volume for the generic method is 5 ul.

The column temperature is 30 deg.

The UV detection range is from 220 to 330 nm.

### Pharmacological Data

Compounds or salts of the invention may be tested for in vitro biological activity at the P2X7 receptor in accordance with the following studies:

### Ethidium Accumulation Assay

Studies were performed using NaCl assay buffer of the following composition: 140 mM NaCl, 10 mM HEPES [4-(2-hydroxyethyl)-1-piperazine-1-ethanesulfonic acid], 5 mM N-methyl-D-glucamine, 5.6 mM KCl, 10 mM D-glucose, 0.5 mM CaCl₂ (pH 7.4).

Human Embryonic Kidney (HEK) 293 cells, stably expressing human recombinant P2X7 receptors, were grown in poly-D-lysine pretreated 96 well plates for 18-24 hours. (The cloning of the human P2X7 receptor is described in U.S. Pat. No. 6,133,434, e.g. see Example 3 therein). The cells were washed twice with 350 μl of the assay buffer, before addition of 50 μl of the assay buffer containing the putative P2X7 receptor antagonist compound. (A small amount of dimethyl sulfoxide, for initially dissolving the compound, is optionally used and present in this 50 μl test compound sample.) The cells were then incubated at room temperature (19-21° C.) for 30 min before addition of ATP and ethidium (100 μM final assay concentration). The ATP concentration was chosen to be close to the EC₈₀ for the receptor type and was 1 mM for studies on the human P2X7 receptor. Incubations were continued for 8 or 16 min and were terminated by addition of 25 μl of 1.3M sucrose containing 4 mM of the P2X7 receptor antagonist Reactive Black 5 (Aldrich). Cellular accumulation of ethidium was determined by measuring fluorescence (excitation wavelength of 530 nm and emission wavelength of 620 nm) from below the plate with a Canberra Packard Fluorocount (14 Station Road, Pangbourne, Reading, Berkshire RG8 7AN, United Kingdom) or a FlexStation II 384 from Molecular Molecular Devices (660-665 Eskdale Road, Wokingham, Berkshire RG41 5TS, United Kingdom). Antagonist pIC₅₀ values for blocking ATP responses were determined using iterative curve fitting techniques.

### Fluorescent Imaging Plate Reader (FLIPR) Ca Assay

Studies were performed using NaCl assay buffer of the following composition for human P2X7: 137 mM NaCl; 20 mM HEPES [4-(2-hydroxyethyl)-1-piperazine-1-ethanesulfonic acid]; 5.37 mM KCl; 4.17 mM NaHCO₃; 1 mM CaCl₂; 0.5 mM MgSO₄; and 1 g/L of D-glucose (pH 7.4).

Human Embryonic Kidney (HEK) 293 cells, stably expressing human recombinant P2X7 receptors, were grown in poly-D-lysine pretreated 384 well plates for 24 hours at room temperature (for a time sufficient for growth of a homogeneous layer of cells at the bottom of the wells). Alternatively, human osteosarcoma (U-20S) cells (commercially available), transduced with modified Baculovirus (BacMam) vector to deliver the gene coding for human P2X7 receptor (i.e. transiently expressing human recombinant P2X7 receptors), were grown in substantially the same conditions as for the HEK293 cells except that the well plates were not pre-treated with poly-D-lysine. (The cloning of the human P2X7 receptor is described in U.S. Pat. No. 6,133,434, e.g. see Example 3 therein). The cells were washed three times with 80 μl of assay buffer, loaded for 1 h at 37° C. with 2 μM Fluo-4-AM [4-(6-acetoxymethoxy-2,7-difluoro-3-oxo-9-xanthenyl)-4′-methyl-2,2′-(ethylenedioxy)dianiline-N,N,N′,N′-tetraacetic acid tetrakis(acetoxymethyl) ester], a Ca²⁺-sensitive, cell-permeable, fluorescent dye (Tef Labs. Inc., 9415 Capitol View Drive, Austin, Tex. 78747, USA), washed three times again (3×80 μl), and left with 30 μl buffer before the addition of 10 μl of the assay buffer containing the putative P2X7 receptor antagonist compound, the compound being added at 4× the final assay concentration chosen. The solution of the putative P2X7 receptor antagonist compound was created by (i) dissolving the compound in dimethyl sulfoxide (DMSO) to create a stock solution in DMSO at 200× the final assay concentration, and (ii) mixing 1 μl of the stock solution of the compound in DMSO with 50 μl of the assay buffer to create a solution at about 4× the final assay concentration. The cells were then incubated at room temperature for 30 mins before addition (online, by FLIPR384 or FLIPR3 instrument (Molecular Devices, 1311 Orleans Drive, Sunnyvale, Calif. 94089-1136, USA)) of benzoylbenzoyl-ATP (BzATP) such as to create a 60 μM final assay concentration of BzATP (BzATP was added at 5× this final concentration). The BzATP concentration was chosen to be close to the EC₈₀ for the receptor type. Incubations and reading were continued for 90 sec, and intracellular calcium increase was determined by measuring fluorescence (excitation wavelength of 488 nm and emission wavelength of 516 nm) from below the plate, with FLIPR charged-coupled device (CCD) camera. Antagonist pIC₅₀ values for blocking BzATP responses were determined using iterative curve fitting techniques.

The compounds of Examples 1, 2, 3, 4, 5, 6, 7, 8, 9, 10, 11 and 12 were tested in the FLIPR Ca Assay (or a slightly modified version thereof) and/or the Ethidium Accumulation Assay (or a slightly modified version thereof) for human P2X7 receptor antagonist activity, and were found to have pIC₅₀ values of about 4.7 or more in the FLIPR Ca Assay (or a slightly modified version thereof), and pIC₅₀ values of >5.5 in the Ethidium Accumulation Assay (or a slightly modified version thereof).

The compounds of Examples 3, 4, 5, 6, 7, 8, 9, 10, 11 and 12 were found to have pIC₅₀ values of from about 6.5 to about 7.5 in the Ethidium Accumulation Assay (or a slightly modified version thereof).

The compound of Example 5 was found to have a pIC₅₀ value of about 7.2-7.3 in the Ethidium Accumulation Assay (or a slightly modified version thereof). In comparison, the separated stereoisomers (with unconfirmed absolute stereochemistry) of the same compound from Example 5A were found to have pIC₅₀ values of about 6.7-6.8 and about 7.3-7.4 respectively in the Ethidium Accumulation Assay (or a slightly modified version thereof).

1-16. (canceled)

17. A pharmaceutical composition comprising a compound of formula (I) or a pharmaceutically acceptable salt thereof: 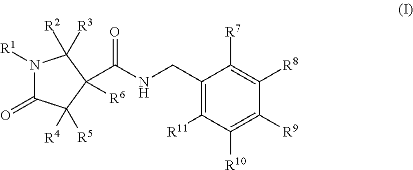
wherein:
R¹ represents C₁₋₄ alkyl, C₂₋₆ alkenyl, C₂₋₆ alkynyl, C₃₋₆ cycloalkyl, C₃₋₆ cycloalkylmethyl-, phenyl-X—, or heteroaryl, any of which may be optionally substituted with C₁₋₆ alkyl, —CF₃, —O—C₁₋₆ alkyl, CN, or 1, 2, or 3 halogen atoms;
X represents —(CR¹²R¹³)ₙ—;
R¹² and R¹³ represent hydrogen or C₁₋₆ alkyl;
n represents an integer selected from 0 to 2;
R², R³, R⁴, R⁵, and R⁶ independently represent hydrogen, fluorine, or methyl; and
R⁷, R⁸, R⁹, R¹⁰, and R¹¹ independently represent hydrogen, halogen, cyano, C₁₋₆ alkyl, C₂₋₆ alkenyl, C₂₋₆ alkynyl, or C₃₋₆ cycloalkyl, wherein any of said C₁₋₆ alkyl, C₂₋₆ alkenyl, C₂₋₆ alkynyl, or C₃₋₆ cycloalkyl groups may be optionally substituted with 1, 2, or 3 halogen atoms, such that at least two of R⁷, R⁸, R⁹, R¹⁰, and R¹¹ represent a group other than hydrogen and at least one of R⁷ and R¹¹ represents a group other than hydrogen.

18. The composition according to claim 17, wherein:
R¹ represents C₁₋₄ alkyl or C₃₋₅ cycloalkyl;
R², R³, R⁴, R⁵, and R⁶ each represent hydrogen;
R⁷ represents hydrogen, fluorine, chlorine, methyl, or —CF₃;
R⁸ represents hydrogen or —CF₃;
R⁹ represents hydrogen, fluorine, or chlorine;
R¹⁰ represents hydrogen, fluorine, chlorine, or —CF₃; and
R¹¹ represents hydrogen, fluorine, chlorine, methyl, or —CF₃;
such that at least two of R⁷, R⁸, R⁹, R¹⁰ and R¹¹ represent a group other than hydrogen and at least one of R⁷ and R¹¹ represents a group other than hydrogen.

19. The composition according to claim 18, wherein:
R¹ represents methyl, ethyl, isobutyl, or cyclopropyl;
R⁷ represents chlorine;
R¹⁰ represents hydrogen; and
R¹¹ represents hydrogen.

20. A compound of formula (IA) or a pharmaceutically acceptable salt thereof: 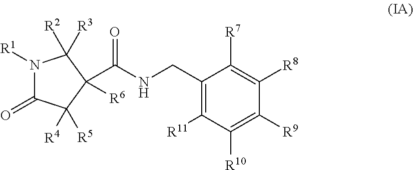
wherein:
R¹ represents C₁₋₄ alkyl, C₂₋₆ alkenyl, C₂₋₆ alkynyl, C₃₋₆ cycloalkyl, C₃₋₆ cycloalkylmethyl-, phenyl-X—, or heteroaryl, any of which may be optionally substituted with C₁₋₆ alkyl, —CF₃, —O—C₁₋₆ alkyl, CN, or 1, 2, or 3 halogen atoms;
X represents —(CR¹²R¹³)ₙ—;
R¹² and R¹³ represent hydrogen or C₁₋₆ alkyl;
n represents an integer selected from 0 to 2;
R², R³, R⁴, R⁵, and R⁶ independently represent hydrogen, fluorine, or methyl; and
R⁷, R⁸, R⁹, R¹⁰, and R¹¹ independently represent hydrogen, halogen, cyano, C₁₋₆ alkyl, C₂₋₆ alkenyl, C₂₋₆ alkynyl, or C₃₋₆ cycloalkyl, wherein any of said C₁₋₆ alkyl, C₂₋₆ alkenyl, C₂₋₆ alkynyl, or C₃₋₆ cycloalkyl groups may be optionally substituted with 1, 2, or 3 halogen atoms, such that at least two of R⁷, R⁸, R⁹, R¹⁰, and R¹¹ represent a group other than hydrogen and at least one of R⁷ and R¹¹ represents a group other than hydrogen,
with the proviso that the compound of formula (IA) is other than: N-[(2,4-dichlorophenyl)methyl]-5-oxo-1-(phenylmethyl)-3-pyrrolidinecarboxamide.

21. The compound or salt according to claim 20, wherein R¹ represents C₁₋₄ alkyl or C₃₋₅ cycloalkyl.

22. The compound or salt according to claim 21, wherein R¹ represents methyl, ethyl, isobutyl, or cyclopropyl.

23. The compound or salt according to claim 20, wherein R², R³, R⁴, R⁵, and R⁶ each represent hydrogen.

24. The compound or salt according to claim 20, wherein:
R⁷ represents hydrogen, halogen, or C₁₋₆ alkyl optionally substituted with 1, 2, or 3 halogen atoms;
R⁸ represents hydrogen or C₁₋₆ alkyl optionally substituted with 1, 2, or 3 halogen atoms;
R⁹ represents hydrogen or halogen;
R¹⁰ represents hydrogen, halogen, or C₁₋₆ alkyl optionally substituted with 1, 2, or 3 halogen atoms; and
R¹¹ represents hydrogen, halogen, or C₁₋₆ alkyl optionally substituted with 1, 2, or 3 halogen atoms;
such that at least two of R⁷, R⁸, R⁹, R¹⁰, and R¹¹ represent a group other than hydrogen and at least one of R⁷ and R¹¹ represents a group other than hydrogen.

25. The compound or salt according to claim 24, wherein:
R⁷ represents hydrogen, fluorine, chlorine, methyl, or —CF₃;
R⁸ represents hydrogen or —CF₃;
R⁹ represents hydrogen, fluorine, or chlorine;
R¹⁰ represents hydrogen, fluorine, chlorine, or —CF₃; and
R¹¹ represents hydrogen, fluorine, chlorine, methyl, or —CF₃;
such that at least two of R⁷, R⁸, R⁹, R¹⁰, and R¹¹ represent a group other than hydrogen and at least one of R⁷ and R¹¹ represents a group other than hydrogen.

26. The compound or salt according to claim 25, wherein:
R⁷ represents chlorine;
R¹⁰ represents hydrogen; and
R¹¹ represents hydrogen.

27. The compound of formula (IA) as defined in claim 20 which is selected from the group consisting of:
N-[(2-chloro-4-fluorophenyl)methyl]-5-oxo-1-[(1R)-1-phenylethyl]-3-pyrrolidinecarboxamide;
N-[(2-Chloro-4-fluorophenyl)methyl]-1-cyclopropyl-5-oxo-3-pyrrolidinecarboxamide;
1-Cyclopropyl-N-[(2,4-dichlorophenyl)methyl]-5-oxo-3-pyrrolidinecarboxamide;
N-{[2-chloro-3-(trifluoromethyl)phenyl]methyl}-1-cyclopropyl-5-oxo-3-pyrrolidinecarboxamide;
N-{[2-chloro-3-(trifluoromethyl)phenyl]methyl}-1-methyl-5-oxo-3-pyrrolidinecarboxamide;
N-[(2,4-dichlorophenyl)methyl]-1-methyl-5-oxo-3-pyrrolidinecarboxamide;
N-[(2,4-dichlorophenyl)methyl]-1-ethyl-5-oxo-3-pyrrolidinecarboxamide;
N-{[2-chloro-3-(trifluoromethyl)phenyl]methyl}-1-ethyl-5-oxo-3-pyrrolidinecarboxamide;
N-{[2-chloro-3-(trifluoromethyl)phenyl]methyl}-1-(2-methylpropyl)-5-oxo-3-pyrrolidinecarboxamide;
N-[(2,4-dichlorophenyl)methyl]-1-(2-methylpropyl)-5-oxo-3-pyrrolidinecarboxamide; and
N-{[2-chloro-3-(trifluoromethyl)phenyl]methyl}-5-oxo-1-(phenylmethyl)-3-pyrrolidinecarboxamide;
or a pharmaceutically acceptable salt thereof.

28. A pharmaceutical composition which comprises the compound or salt as defined in claim 20, and a pharmaceutically acceptable carrier or excipient.

29. A method of treating a human suffering from pain, rheumatoid arthritis, or osteoarthritis, which method comprises administering to said human an effective amount of the compound or salt as defined in claim 20.

30. A method of treating a human suffering from Alzheimer's disease or mild cognitive impairment associated with aging, which method comprises administering to said human an effective amount of the compound or salt as defined in claim 20.

31. A method of treating a human suffering from senile dementia, dementia with Lewy bodies, Pick's disease, Huntingdon's chorea, Parkinson's disease Creutzfeldt-Jakob disease, Amyotrophic Lateral Sclerosis, motor neuron disease, or multi-infarct dementia, which method comprises administering to said human an effective amount of the compound or salt as defined in claim 20.
